(19) United States
(12) Patent Application Publication
(10) Pub. No.: US 2023/0306553 A1
(43) Pub. Date: Sep. 28, 2023

(54) MITIGATING COMPRESSION INDUCED LOSS OF INFORMATION IN TRANSMITTED IMAGES

(71) Applicant: INTERNATIONAL BUSINESS MACHINES CORPORATION, ARMONK, NY (US)

(72) Inventors: Andrew Jason Lavery, Austin, TX (US); Henry Feldman, Needham, MA (US)

(21) Appl. No.: 17/656,013

(22) Filed: Mar. 23, 2022

(51) Int. Cl. G06T 3/40; G06T 9/00; G16H 30/40; H04N 1/00

(52) U.S. Cl. CPC G06T 3/40; G06T 9/00; G16H 30/40; H04N 1/00031; H04N 1/0001

(57) ABSTRACT

Disclosed are techniques for mitigating loss of information in electronically transmitted images caused by compression operations performed on the images to facilitate transmissions. When an image for electronic transmission is received, a computer vision model extracts various points of data from the image corresponding to information present in the image that is intended for human consumption (for example, in a scan of a handwritten note from a doctor prescribing a medicine for a patient, some of the data points may include the name of the medicine, the dosage value, and when the medicine should be consumed). A test transmission image is then generated by applying the compression operations which are applied in the electronic transmission to a copy of the image, and that copy is also inputted to the computer vision model for data point extraction. Differences in the extracted data points are used to modify the image for transmission.

### BACKGROUND

The present invention relates generally to the field of machine learning, and more particularly to mitigating information loss from compression when transmitting images.

Machine learning (ML) is the study of computer algorithms which automatically improve through experience. It is typically viewed as a subset of artificial intelligence (AI). Machine learning algorithms typically construct a mathematical model based on sample data, sometimes known as “training data”, in order to determine predictions or decisions without being specifically programmed to do so. Typically, machine learning models require a large quantity of data in order for them to perform well. Often, when training a machine learning model, one needs to collect a large, representative sample of data from a given training set. Data from the training set can be as varied as a corpus of text, a collection of images (or videos), and data collected from individual users of a service.

The Wikipedia entry for “digital image” (as of Feb. 23, 2022) states as follows: “A digital image is an image composed of picture elements, also known as pixels, each with finite, discrete quantities of numeric representation for its intensity or gray level that is an output from its two-dimensional functions fed as input by its spatial coordinates denoted with x, y on the x-axis and y-axis, respectively. Depending on whether the image resolution is fixed, it may be of vector or raster type.”

The Wikipedia entry for “Image compression” (as of Feb. 23, 2022) states as follows: “Image compression is a type of data compression applied to digital images, to reduce their cost for storage or transmission. Algorithms may take advantage of visual perception and the statistical properties of image data to provide superior results compared with generic data compression methods which are used for other digital data . . . . Image compression may be lossy or lossless. Lossless compression is preferred for archival purposes and often for medical imaging, technical drawings, clip art, or comics. Lossy compression methods, especially when used at low bit rates, introduce compression artifacts.”

The Wikipedia entry for “Fax” (as of Feb. 23, 2022) states as follows: “Fax (short for facsimile), sometimes called telecopying or telefax (the latter short for telefacsimile), is the telephonic transmission of scanned printed material (both text and images), normally to a telephone number connected to a printer or other output device. The original document is scanned with a fax machine (or a telecopier), which processes the contents (text or images) as a single fixed graphic image, converting it into a bitmap, and then transmitting it through the telephone system in the form of audio-frequency tones. The receiving fax machine interprets the tones and reconstructs the image, printing a paper copy. Early systems used direct conversions of image darkness to audio tone in a continuous or analog manner. Since the 1980s, most machines modulate the transmitted audio frequencies using a digital representation of the page which is compressed to quickly transmit areas which are all-white or all-black.”

The Wikipedia entry for “Optical character recognition” (as of Feb. 23, 2022) states as follows: “Optical character recognition or optical character reader (OCR) is the electronic or mechanical conversion of images of typed, handwritten or printed text into machine-encoded text, whether from a scanned document, a photo of a document, a scene-photo (for example the text on signs and billboards in a landscape photo) or from subtitle text superimposed on an image (for example: from a television broadcast). Widely used as a form of data entry from printed paper data records—whether passport documents, invoices, bank statements, computerized receipts, business cards, mail, printouts of static-data, or any suitable documentation—it is a common method of digitizing printed texts so that they can be electronically edited, searched, stored more compactly, displayed on-line, and used in machine processes such as cognitive computing, machine translation, (extracted) text-to-speech, key data and text mining. OCR is a field of research in pattern recognition, artificial intelligence and computer vision. Early versions needed to be trained with images of each character, and worked on one font at a time. Advanced systems capable of producing a high degree of recognition accuracy for most fonts are now common, and with support for a variety of digital image file format inputs. Some systems are capable of reproducing formatted output that closely approximates the original page including images, columns, and other non-textual components.”

Computer vision is an interdisciplinary field which grapples with how computers can be granted the ability to gain high-level understanding from digital images or videos. From an engineering perspective, it seeks to automate tasks that the human visual system can do. Computer vision related to the automatic extraction, analysis and understanding of useful information from a single image or a sequence of images such as an animation or video feed. It involves developing a theoretical and algorithmic basis to achieve automatic visual understanding.

Medical records refers to documents, images, or any other records of information corresponding to medical status of a patient, including any procedures received, tests and their corresponding results, or any information pertaining to the medical history of a patient.

### SUMMARY

According to an aspect of the present invention, there is a method, computer program product and/or system that performs the following operations (not necessarily in the following order): (i) receiving an original image for electronic transmission, with the electronic transmission having a compression transformation applied to transmitted images; (ii) determining, with a computer vision machine learning model, an original set of data values corresponding to a human understanding of information present in the original image; (iii) generating a test transmission image corresponding to a resulting image from application of the compression transformation to the original image; (iv) determining, with the computer vision machine learning model, a test set of data values corresponding to a human understanding of information present in the test transmission image; and (v) determining whether there is information loss induced by transmission of the original image by comparing the original set of data values and the test set of data values for differences.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a screenshot view generated by the first embodiment system;

FIG. 5 is block diagram view of a second embodiment system according to the present invention;

FIG. 6 is a screenshot view generated by the second embodiment system; and

FIG. 7 is a screenshot view generated by the second embodiment system.

### DETAILED DESCRIPTION

Some embodiments of the present invention are directed to techniques for mitigating loss of information in electronically transmitted images caused by compression operations performed on the images to facilitate transmissions. When an image for electronic transmission is received, a computer vision model extracts various points of data from the image corresponding to information present in the image that is intended for human consumption (for example, in a scan of a handwritten note from a doctor prescribing a medicine for a patient, some of the data points may include the name of the medicine, the dosage value, and when the medicine should be consumed). A test transmission image is then generated by applying the compression operations which are applied in the electronic transmission to a copy of the image, and that copy is also inputted to the computer vision model for data point extraction. Differences in the extracted data points are used to modify the image for transmission.

This Detailed Description section is divided into the following subsections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium (sometimes referred to as “machine readable storage medium”) can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

A “storage device” is hereby defined to be any thing made or adapted to store computer code in a manner so that the computer code can be accessed by a computer processor. A storage device typically includes a storage medium, which is the material in, or on, which the data of the computer code is stored. A single “storage device” may have: (i) multiple discrete portions that are spaced apart, or distributed (for example, a set of six solid state storage devices respectively located in six laptop computers that collectively store a single computer program); and/or (ii) may use multiple storage media (for example, a set of computer code that is partially stored in as magnetic domains in a computer's non-volatile storage and partially stored in a set of semiconductor switches in the computer's volatile memory). The term “storage medium” should be construed to cover situations where multiple different types of storage media are used.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

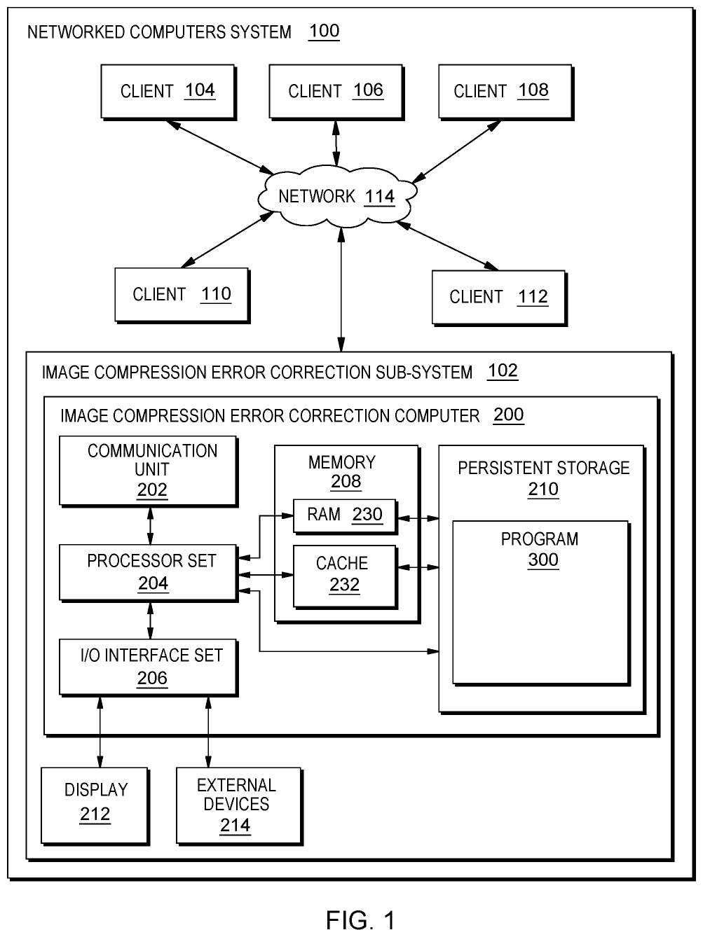

As shown in FIG. 1, networked computers system 100 is an embodiment of a hardware and software environment for use with various embodiments of the present invention, described in detail with reference to the Figures. Networked computers system 100 includes: server subsystem 102 (sometimes herein referred to, more simply, as subsystem 102); client subsystems 104, 106, 108, 110, 112; and communication network 114. Server subsystem 102 includes: server computer 200; communication unit 202; processor set 204; input/output (I/O) interface set 206; memory 208; persistent storage 210; display 212; external device(s) 214; random access memory (RAM) 230; cache 232; and program 300.

Subsystem 102 may be a laptop computer, tablet computer, netbook computer, personal computer (PC), a desktop computer, a personal digital assistant (PDA), a smart phone, or any other type of computer (see definition of “computer” in Definitions section, below). Program 300 is a collection of machine readable instructions and/or data that is used to create, manage and control certain software functions that will be discussed in detail, below, in the Example Embodiment subsection of this Detailed Description section.

Subsystem 102 is capable of communicating with other computer subsystems via communication network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client subsystems.

Subsystem 102 is shown as a block diagram with many double arrows. These double arrows (no separate reference numerals) represent a communications fabric, which provides communications between various components of subsystem 102. This communications fabric can be implemented with any architecture designed for passing data and/or control information between processors (such as microprocessors, communications and network processors, etc.), system memory, peripheral devices, and any other hardware components within a computer system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for subsystem 102; and/or (ii) devices external to subsystem 102 may be able to provide memory for subsystem 102. Both memory 208 and persistent storage 210: (i) store data in a manner that is less transient than a signal in transit; and (ii) store data on a tangible medium (such as magnetic or optical domains). In this embodiment, memory 208 is volatile storage, while persistent storage 210 provides nonvolatile storage. The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202 provides for communications with other data processing systems or devices external to subsystem 102. In these examples, communications unit 202 includes one or more network interface cards. Communications unit 202 may provide communications through the use of either or both physical and wireless communications links. Any software modules discussed herein may be downloaded to a persistent storage device (such as persistent storage 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. I/O interface set 206 also connects in data communication with display 212. Display 212 is a display device that provides a mechanism to display data to a user and may be, for example, a computer monitor or a smart phone display screen.

In this embodiment, program 300 is stored in persistent storage 210 for access and/or execution by one or more computer processors of processor set 204, usually through one or more memories of memory 208. It will be understood by those of skill in the art that program 300 may be stored in a more highly distributed manner during its run time and/or when it is not running. Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

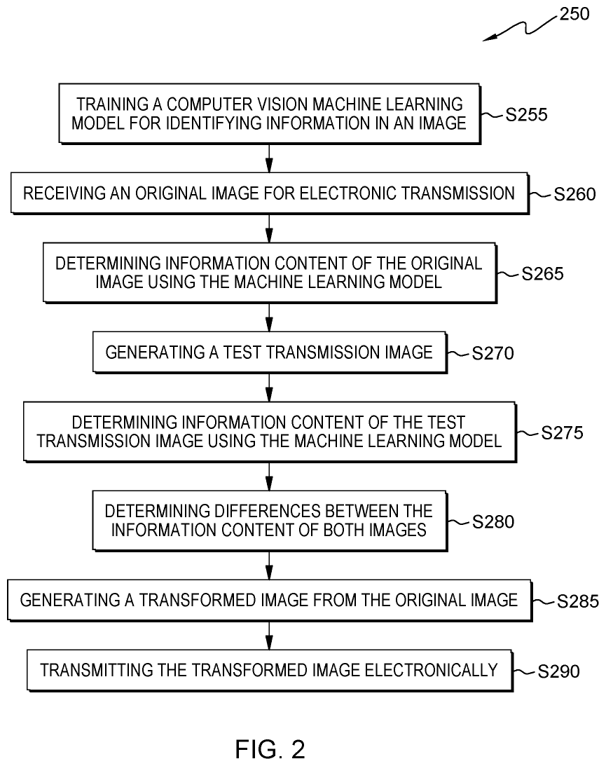

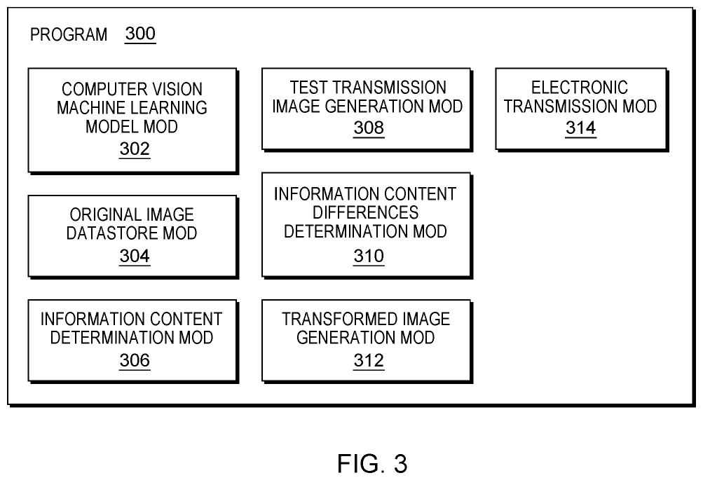

As shown in FIG. 1, networked computers system 100 is an environment in which an example method according to the present invention can be performed. As shown in FIG. 2, flowchart 250 shows an example method according to the present invention. As shown in FIG. 3, program 300 performs or control performance of at least some of the method operations of flowchart 250. This method and associated software will now be discussed, over the course of the following paragraphs, with extensive reference to the blocks of FIGS. 1, 2, 3 and 4.

Processing begins at operation S255, where computer vision machine learning module (“mod”) 302 trains a computer vision machine learning model for identifying information in an image. In this simplified embodiment, the computer vision machine learning model is trained using known computer vision and optical character recognition techniques to determine a set of information from an input image corresponding to human understanding of the image. For example, determining text information present in the image, colors, languages present, font sizes, etc. More specifically, this computer vision machine learning model outputs a dataset of information with entries corresponding to various information datapoints with each entry including a set of pixels corresponding to where in the image the entry is sourced from. In some alternative embodiments, the computer vision machine learning model identifies entries corresponding to medical information present in the image (such as medicine dosage instructions or lab test results). In some alternative embodiments, this computer vision machine learning model is trained using a dataset of images and corresponding labels, with the corresponding labels including a set of datapoints of human understandable information and their corresponding pixels in a given image. For example, a first datapoint, datapoint 1, includes the following information: (i) data type=medication name; (ii) data value=aspirin; and (iii) image location=pixels 100,100 through 700, 300. In another example, a second datapoint, datapoint 2, includes the following information: (i) data type=glucose test; (ii) data value=green; and (iii) image location=pixels 250,100 through 555, 342. In yet another example, a third datapoint, datapoint 3, includes the following information: (i) data type=dosage instructions; (ii) data value=every day; and (iii) image location=pixels 354,408 through 861,626.

Processing proceeds to operation S260, where original image datastore mod 304 receives an original image for electronic transmission. In this simplified embodiment, the original image is a scanned image of results of a blood test taken for patient John Smith. This image, the original image, is being electronically transmitted via fax transmission from healthcare provider A, with whom client 104 is a computer device associated with, to healthcare provider B, with whom client 106 is a computer device associated with. The image includes the following information: (i) a test results legend showing: (a) normal results are shown with a corresponding green square (a square colored with the color green), (b) moderately abnormal results are shown with a corresponding yellow square (a square colored with the color yellow), and (c) severely abnormal results are shown with a corresponding red square (a square colored with the color red); (ii) a green square corresponding to a first blood test result for glucose; and (iii) a yellow square corresponding to a second blood test result for creatinine. The fax transmission process that is used to transmit the original image includes image compression processes which compress the image prior to transmission to the receiver, which then prints a copy of the transmitted compressed image.

Processing proceeds to operation S265, where information content determination mod 306 determines information content of the original image using the machine learning model. In this simplified embodiment, the original image stored in original image datastore mod 304 is provided as input to the computer vision machine learning model, trained at S255. This machine learning model then processes the original image to determine the information content of the original image. For example, in this simplified embodiment, the computer vision machine learning model determines the following information from the original image, in a set of datapoints: (i) datapoint 1; (ii) datapoint 2; (iii) datapoint 3; (iv) datapoint 4; and (v) datapoint 5. For datapoint 1, the computer vision machine learning model determines the following information: (i) data type=“normal”; (ii) data value=green; (iii) image location=pixels 300,300 through 400,350. For datapoint 2, the computer vision machine learning model determines the following information: (i) data type=“moderately abnormal”; (ii) data value=yellow; (iii) image location=pixels 300,450 through 400,500. For datapoint 3, the computer vision machine learning model determines the following information: (i) data type=“severely abnormal”; (ii) data value=red; (iii) image location=pixels 300,600 through 400,650. For datapoint 4, the computer vision machine learning model determines the following information: (i) data type=“glucose”; (ii) data value=green; (iii) image location=pixels 300,850 through 400,900. For datapoint 5, the computer vision machine learning model determines the following information: (i) data type=“creatinine”; (ii) data value=yellow; (iii) image location=pixels 300,1000 through 400,1050. In some alternative embodiments, the datapoints may contain additional details, such as the shape and RGB color values (short for red, green, blue) or hexadecimal code color values of data values. Further, the datapoints may specify what kind of information is present in the data value. For example, either: (i) data value=color, red; or (ii) data value=text, john smith. In the two immediately preceding examples of these alternative embodiments, the data value field has two subfields: (i) a kind subfield (such as color or text in these two examples); and (ii) a value subfield (such as red or john smith in these two examples.)

Processing proceeds to operation S270, where test transmission image generation mod 308 generates a test transmission image. In this simplified embodiment, test transmission image generation mod 308 generates the test transmission image from processing a copy of the original image stored in original image datastore mod 304 with the image compression that would apply to the original image when transmitted electronically, resulting in a test transmission image that is a compressed copy of the original image. Notably, this compression includes transforming all colors present in the original image into greyscale, or shades of grey between white and black. The test transmission image is otherwise similar to the original image, including similar text information present, though subjected to some compression which may adjust the appearance of said text information (and depending on the language or font size, this can affect what words are interpreted by a reader when reading the text).

Processing proceeds to operation S275, where information content determination mod 306 determines information content of the test transmission image using the machine learning model. In this simplified embodiment, the test transmission image generated at S270 is provided as input to the computer vision machine learning model, which was trained at S255. This machine learning model then processes the test transmission image to determine the information content of the test transmission image. For example, in this simplified embodiment, the computer vision machine learning model determines the following information from the test transmission image, in a set of datapoints: (i) test transmission datapoint 1; (ii) test transmission datapoint 2; (iii) test transmission datapoint 3; (iv) test transmission datapoint 4; and (v) test transmission datapoint 5. For test transmission datapoint 1, the computer vision machine learning model determines the following information: (i) data type=“normal”; (ii) data value=dark gray; (iii) image location=pixels 300,300 through 400,350. For test transmission datapoint 2, the computer vision machine learning model determines the following information: (i) data type=“moderately abnormal”; (ii) data value=light gray; (iii) image location=pixels 300,450 through 400,500. For test transmission datapoint 3, the computer vision machine learning model determines the following information: (i) data type=“severely abnormal”; (ii) data value=gray; (iii) image location=pixels 300,600 through 400,650. For test transmission datapoint 4, the computer vision machine learning model determines the following information: (i) data type=“glucose”; (ii) data value=dark gray; (iii) image location=pixels 300,850 through 400,900. For test transmission datapoint 5, the computer vision machine learning model determines the following information: (i) data type=“creatinine”; (ii) data value=light gray; (iii) image location=pixels 300,1000 through 400,1050.

Processing proceeds to operation S280, where information content differences determination mod 310 determines differences between the information content of both images. In this simplified embodiment, information content differences determination mod 310 compares the data types and data values of the datapoints in the information content of the original image and the information content of the test transmission image, using the image locations of the datapoints to determine which datapoints to use in comparisons. For example, in this simplified embodiment, each of the datapoints in the information content of the original image is determined to have different information in the test transmission image, as follows.

Datapoint 1, corresponding to the first datapoint of the original image, is compared to test transmission datapoint 1, based on their similar image locations (or locations within their respective images) of pixels 300,300 through 400,350; while datapoint 1 and test transmission datapoint 1 have equivalent information for data type (both of them have “normal” for their data type), test transmission datapoint 1 differs from datapoint 1 [of the original image] in their respective data values, where test transmission datapoint 1 has a data value of “dark gray” and datapoint 1 [of the original image] has a data value of “green.”

Datapoint 2, corresponding to the second datapoint of the original image, is compared to test transmission datapoint 2, based on their similar image locations (or locations within their respective images) of pixels 300,450 through 400,500; while datapoint 2 and test transmission datapoint 2 have equivalent information for data type (both of them have “moderately abnormal” for their data type), test transmission datapoint 2 differs from datapoint 2 [of the original image] in their respective data values, where test transmission datapoint 2 has a data value of “light gray” and datapoint 2 [of the original image] has a data value of “yellow.”

Datapoint 3, corresponding to the third datapoint of the original image, is compared to test transmission datapoint 3, based on their similar image locations (or locations within their respective images) of pixels 300,600 through 400,650; while datapoint 3 and test transmission datapoint 3 have equivalent information for data type (both of them have “severely abnormal” for their data type), test transmission datapoint 3 differs from datapoint 3 [of the original image] in their respective data values, where test transmission datapoint 3 has a data value of “gray” and datapoint 3 [of the original image] has a data value of “red.”

Datapoint 4, corresponding to the fourth datapoint of the original image, is compared to test transmission datapoint 4, based on their similar image locations (or locations within their respective images) of pixels 300,850 through 400,900; while datapoint 4 and test transmission datapoint 4 have equivalent information for data type (both of them have “glucose” for their data type), test transmission datapoint 4 differs from datapoint 4 [of the original image] in their respective data values, where test transmission datapoint 4 has a data value of “dark gray” and datapoint 4 [of the original image] has a data value of “green.”

Datapoint 5, corresponding to the fifth datapoint of the original image, is compared to test transmission datapoint 5, based on their similar image locations (or locations within their respective images) of pixels 300,1000 through 400,1050; while datapoint 5 and test transmission datapoint 5 have equivalent information for data type (both of them have “creatinine” for their data type), test transmission datapoint 5 differs from datapoint 5 [of the original image] in their respective data values, where test transmission datapoint 5 has a data value of “light gray” and datapoint 5 [of the original image] has a data value of “yellow.”

Further, if there is no datapoint in the information content of the test transmission image that is suitable for comparison to a given datapoint of the information content of the original image (for example, a datapoint with a similar image location is not found), the given datapoint of the information content of the original image is flagged as having a difference in the test transmission. The change of colors to grayscale to facilitate the electronic transmission induces a loss of information from how a human would interpret the contents of the document —green, yellow and red are readily distinguishable by most individuals, but many would be challenged to quickly differentiate between different shades of gray. Other types of induced information loss that would be reflected by comparing the test transmission image to the original image to identify ambiguous letters or numbers. For example, if compression artifacts are present on small font, a computer vision model (and also a human) may have difficulty correctly identifying letters that may appear similar in many fonts (such as “nn” and “m,” “O” and “0,” “vv” and “w,” “I” and “l,” etc.) These letters, which may be more clearly distinguishable in the original image, are often easy to misinterpret with compression artifacts.

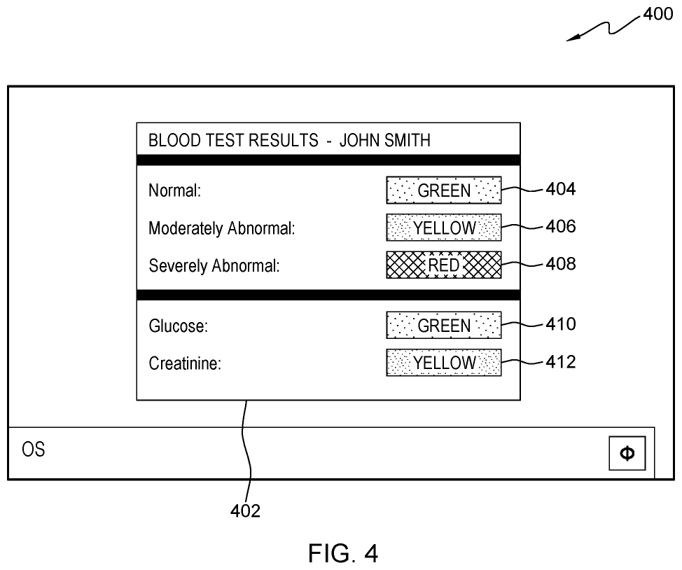

Processing proceeds to operation S285, where transformed image generation mod 312 generates a transformed image from the original image. In this simplified embodiment, transformed image generation mod 312 generates the transformed image from the original image by enhancing a copy of the original image with annotations in the transformed image. These annotations correspond to the differences between the original image and the test transmission image determined at S280. More specifically, these annotations include labels describing datapoints in the information content of the original image that are either different or not found in the test transmission image. For example, in this simplified embodiment, the transformed image is shown in screenshot 400 of FIG. 4, including: (i) transformed image 402; (ii) labeled datapoint 1 data value 404; (iii) labeled datapoint 2 data value 406; (iv) labeled datapoint 3 data value 408; (v) labeled datapoint 4 data value 410; and (vi) labeled datapoint 5 data value 412. As discussed above in the proceeding operations, in this simplified embodiment the data values for each of the five datapoints were found to have different values between the original image and the test transmission image. As such, each is labeled with an annotation describing what information was determined by the computer vision machine learning model determined to be present in the original image corresponding to that datapoint, with the annotation positioned in the transformed image using the image location information for that datapoint in the original image.

In some alternative embodiments, other types of transformations that transformed image generation mod 312 can use to generate the transformed image from the original image includes: (i) increasing the font size of text; (ii) changing the font type; and (iii) changing the color of the text (such as changing the color along the greyscale to increase contrast with the background color of the fax). In yet further embodiments, the type of transformation applied is determined by transformed image generation mod 312 based on the kinds of differences determined at S280. For example, using the data value subfields described in some previously discussed alternative embodiments, if the kind subfield indicates text, then annotation would not be appropriate to preserve information (annotating text with additional text repeating the same information would confuse instead of clarify), so transformed image generation mod 312 increases the font size of the text corresponding to the datapoints where there were differences between the original image and the test transformation image (and/or other appropriate transformations, such as changing the font of the text to one that is preserved better through compression.) Alternatively, if the kind subfield indicates color, then changing the font size would not be appropriate (or changing the font type, or magnification of the portion of the original image), as there is no text to modify or magnify. As a further alternative, font size increase is applied to all the text in the original image for consistency.

Processing proceeds to operation S290, where electronic transmission mod 314 transmits the transformed image electronically. In this simplified embodiment, electronic transmission mod 314 transmits the transformed image electronically by faxing the transformed image to client 106, instead of faxing the original image received at S260. In some alternative embodiments, the electronic transmission corresponds to any kind of electronic transmission for transmitting images electronically between two electronic devices, where such transmission includes compression applied to the contents of the images. In some alternative embodiments, the transformed image corresponds to a set of images of digitally magnified portions of the original image where there were differences found in the test transmission image, which are then subsequently transmitted electronically with the original image. In yet other alternative embodiments, responsive to determining that there are differences between the information contents of original image and the test transmission image, compression is disabled for transmitting the original and/or transformed image. In some of these alternative embodiments, in lieu of generating a transformed image, compression of the original image is disabled when transmitting the original image electronically.

### III. Further Comments and/or Embodiments

Some embodiments of the present invention recognize the following facts, potential problems and/or potential areas for improvement with respect to the current state of the art: (i) many medical records are faxed from one provider to another, from a provider to an insurance company, or between other parties; (ii) those faxes are transmitted as images, and there can be a loss of information in the receiving side of the fax; (iii) the fax compresses the image at the sending side, and decompresses the same at the receiving side, in order to send the fax more quickly; (iv) unfortunately, this can sometimes lead to unusable documents on the receiving side, or worse, it could result in a medical error by using the image on the receiving side; and (v) there exists a need for an approach to reduce or eliminate that loss of information, while still allowing for image compression and faxing of medical documents.

Some embodiments of the present invention may include one, or more, of the following operations, features, characteristics and/or advantages: (i) a system to detect and correct for image compression errors in medical document sharing; (ii) a software module that analyzes potential source documents that are going to be faxed or transferred remotely to optimize the compression while preserving medical meaning; (iii) attempt one or more compression or transformation techniques; (iv) then attempt to decompress the resulting encoding to see if the original image can be derived with enough fidelity to not lose data; (v) use state of the art techniques to detect language (e.g. Arabic); (vi) based on the result of those techniques, optimize the image compression in certain sensitive languages (Arabic); (vii) or eliminate the compression entirely for the given image; (viii) the text is scanned for problematic words (like the earlier Arabic example); (ix) if problematic words are found, eliminate compression for the image or scale the text in the image to appear larger prior to compression; (x) if no problematic words are found, default compression is used; (xi) in another technique for determining appropriate compression, send a sample fax to a test system; (xii) decode the received fax with OCR; (xiii) see if we get the same results as OCR on the original fax; (xiv) if not, adjust compression, scale, or annotate as needed; (xv) source documents that contain certain features (e.g. colors) could be recognized, and annotations could augment those documents; and (xvi) this can include medical notation, such as color coding lab values that are out of range, and annotating those to ensure that a faxed version does not lose any important medical notation.

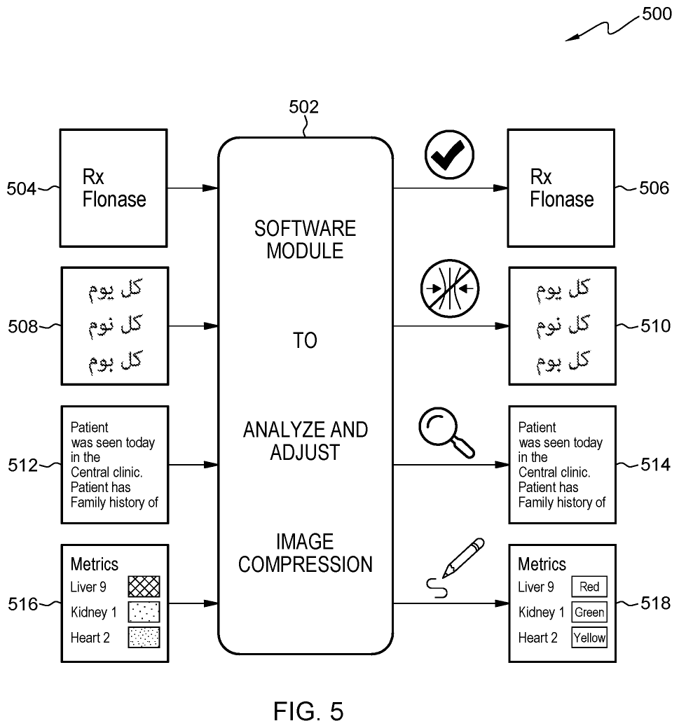

Block diagram 500 of FIG. 5 shows an embodiment of the present invention for analyzing and adjusting image compression for images to preserve information when transmitting said images, including: (i) software module 502; (ii) first original image 504; (iii) corresponding first transmitted image 506; (iv) second original image 508; (v) corresponding second transmitted image 510; (vi) third original image 512; (vii) corresponding third transmitted image 514; (viii) fourth original image 516; and (ix) corresponding fourth transmitted image 518. In this embodiment, first original image 504 is transmitted according to default transmission processes without software module 502 applying any changes to the transmission processes. Software module 502, upon detecting the presence of Arabic language in second original image 508, transmits second original image 508 without compression, resulting in corresponding second image 510. Software module 502, upon detecting a font size below a threshold in third original image 512, transmits third original image 512 with increased font size on the portions with font size below the threshold, resulting in corresponding third image 514. Software module 502, upon detecting non-grayscale colors in fourth original image 516, transmits fourth original image 516 with annotations labeling the colors in fourth original image 516, resulting in corresponding fourth image 518 with the annotations present.

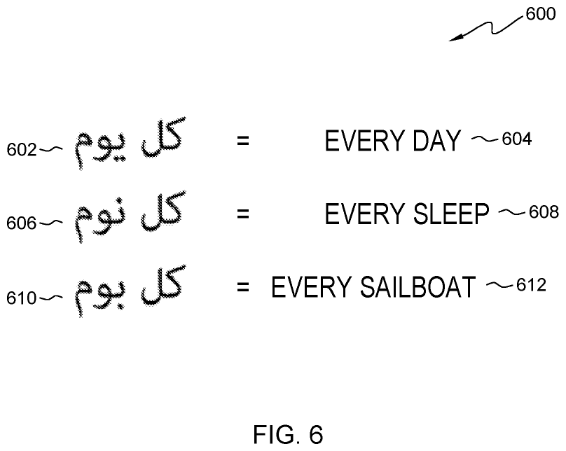

Screenshot 600 of FIG. 6 shows a set of Arabic phrases and their corresponding definitions in English, highlighting the subtle differences in the Arabic phrases which are prone to confusion when subjected to image compression. Arabic phrase 602 corresponds to English phrase 604. Arabic phrase 606 corresponds to English phrase 608. Arabic phrase 610 corresponds to English phrase 612. The only difference between Arabic phrase 602, 606 and 610 is the accent on the first letter of the second word.

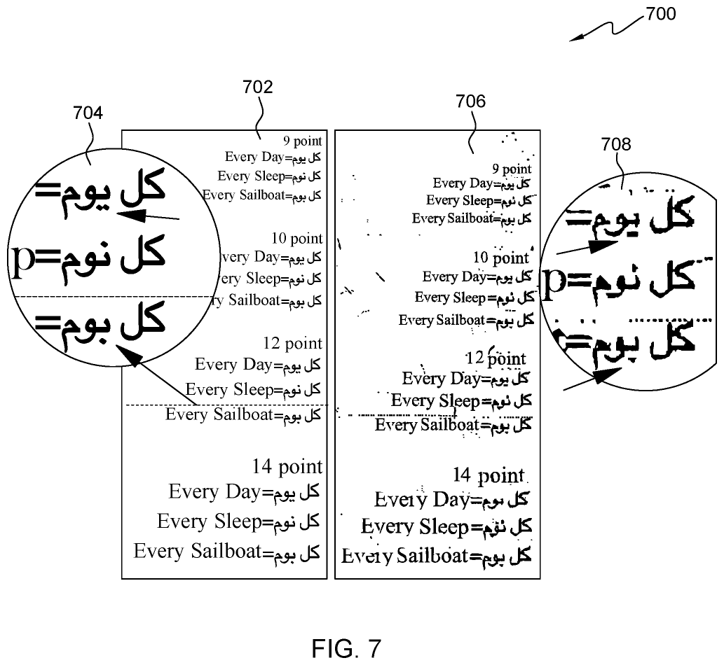

Screenshot 700 of FIG. 7 shows an original image 702 prior to transmission and a corresponding transmitted image 706, which has been subjected to typical image compression accompanying typical electronic transmission. Original image magnification 704, when compared to transmitted image magnification 708, illustrates information loss which occurs as a result of image compression, highlighting how accents on letters in the Arabic language become difficult to distinguish from one another when subjected to image compression.

According to an aspect of the present invention, there is a method, computer program product and/or system for preventing meaning changes due to transmission or compression errors in transmitted documents that performs the following operations (not necessarily in the following order): (i) receiving an original document targeted for transmission; (ii) analyzing the original document for susceptibility to transmission error resulting in a meaning change; and (iii) performing an action to prevent a utilization of the meaning change.

The method, computer program product and/or system of the previous paragraph may further include one, or more, of the following operations, features, characteristics and/or advantages: (i) the action is adjusting the original document; (ii) training a transmission model tailored to a data model to identify data elements susceptible to being transformed due to the transmission to other data elements in modified documents; (iii) examining the original document based on the transmission model by substituting the identified elements susceptible to being transformed into the other elements to form a set of modified elements in the modified documents; (iv) analyzing the data elements modified in the document for meaningful changes to identify locations with elements [words, artifacts] susceptible to meaning changes; (v) annotating the original document at the identified locations indicating possible meaning changes; (vi) the data elements are letters in a language; (vii) the data elements are schematic elements in a drawing; (viii) the transmission model is utilized to mitigate the effect of the transmission [e.g. scaling at least a portion of the document]; (ix) the action is annotating the document to identify features expected to be lost during the transmission [e.g., color in a black and white fax transmission]; (x) the transmission model further comprises sending a test transmission to form a test output; (xi) the transmission model further comprises applying optical character recognition to the test output to form a text representation; (xii) the transmission model further comprises comparing the text representation to the test transmission to form a basis for identifying potential problems; (xiii) the document has critical information [undue risk of bad effects]; and (xiv) the critical information is selected from a group consisting of medical records, flight plans, engineering documents, and financial transactions.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

In an Including/include/includes: unless otherwise explicitly noted, means “including but not necessarily limited to.”

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, and application-specific integrated circuit (ASIC) based devices.

We: this document may use the word “we,” and this should be generally be understood, in most instances, as a pronoun style usage representing “machine logic of a computer system,” or the like; for example, “we processed the data” should be understood, unless context indicates otherwise, as “machine logic of a computer system processed the data”; unless context affirmatively indicates otherwise, “we,” as used herein, is typically not a reference to any specific human individuals or, indeed, and human individuals at all (but rather a computer system).

What is claimed is:

1. A computer-implemented method (CIM) comprising:
receiving an original image data set corresponding to an original image for electronic transmission that will include application of a transformation to compress data as the data is being transmitted;
determining, with a computer vision machine learning (ML) model, an original set of ML output values that represent a human understanding of information present in the original image;
applying the transformation to the original image data set to obtain a test transmission image data set corresponding to a transmission image;
determining, with the computer vision machine learning (ML) model, a transmission test set of ML output values that represent a human understanding of information present in the transmission image; and
comparing the transmission test set of ML output values with the original set of ML output values for differences to determine that an unacceptable degree of information loss is occurring in the electronic transmission of images used in connection with the computer vision ML model.

2. The CIM of claim 1, further comprising:
responsive to determining that an unacceptable degree of information loss is occurring in the electronic transmission of images used in connection with the computer vision ML model, transmitting the original image to a receiver electronic device through the electronic transmission, where the transformation to compress data is disabled for transmission of the original image.

3. The CIM of claim 1, further comprising:
responsive to determining that an unacceptable degree of information loss is occurring in the electronic transmission of images used in connection with the computer vision ML model, determining a set of modifications to the original image based, at least in part, on differences between the original set of ML output values and the test set of ML output values.

4. The CIM of claim 3, further comprising:
generating a modified image for electronic transmission corresponding to the original image transformed with the set of modifications; and
transmitting the modified image to a receiver electronic device through electronic transmission.

5. The CIM of claim 3, wherein the set of modifications are selected from the group consisting of: scaling up segments of text to a larger font size when data values corresponding to those segments of text in the original image and the test image are determined differently by the computer vision ML model, magnifying portions of the original image when data values corresponding to those portions of the original image and test image are determined differently by the computer vision ML model, and annotating text upon elements in the image when data values corresponding to those elements of the original image and the test image are determined differently by the computer vision ML model.

6. The CIM of claim 1, wherein:
the original image corresponds to medical information; and
the electronic transmission corresponds to a fax transmission.

7. A computer program product (CPP) comprising:
a machine readable storage device; and
computer code stored on the machine readable storage device, with the computer code including instructions for causing a processor(s) set to perform operations including the following:
receiving an original image data set corresponding to an original image for electronic transmission that will include application of a transformation to compress data as the data is being transmitted,
determining, with a computer vision machine learning (ML) model, an original set of ML output values that represent a human understanding of information present in the original image,
applying the transformation to the original image data set to obtain a test transmission image data set corresponding to a transmission image,
determining, with the computer vision machine learning (ML) model, a transmission test set of ML output values that represent a human understanding of information present in the transmission image, and
comparing the transmission test set of ML output values with the original set of ML output values for differences to determine that an unacceptable degree of information loss is occurring in the electronic transmission of images used in connection with the computer vision ML model.

8. The CPP of claim 7, wherein the computer code further includes instructions for causing the processor(s) set to perform the following operations:
responsive to determining that an unacceptable degree of information loss is occurring in the electronic transmission of images used in connection with the computer vision ML model, transmitting the original image to a receiver electronic device through the electronic transmission, where the transformation to compress data is disabled for transmission of the original image.

9. The CPP of claim 7, wherein the computer code further includes instructions for causing the processor(s) set to perform the following operations:
responsive to determining that an unacceptable degree of information loss is occurring in the electronic transmission of images used in connection with the computer vision ML model, determining a set of modifications to the original image based, at least in part, on differences between the original set of ML output values and the test set of ML output values.

10. The CPP of claim 9, wherein the computer code further includes instructions for causing the processor(s) set to perform the following operations:
generating a modified image for electronic transmission corresponding to the original image transformed with the set of modifications; and
transmitting the modified image to a receiver electronic device through electronic transmission.

11. The CPP of claim 9, wherein the set of modifications are selected from the group consisting of: scaling up segments of text to a larger font size when data values corresponding to those segments of text in the original image and the test image are determined differently by the computer vision ML model, magnifying portions of the original image when data values corresponding to those portions of the original image and test image are determined differently by the computer vision ML model, and annotating text upon elements in the image when data values corresponding to those elements of the original image and the test image are determined differently by the computer vision ML model.

12. The CPP of claim 7, wherein:
the original image corresponds to medical information; and
the electronic transmission corresponds to a fax transmission.

13. A computer system (CS) comprising:
a processor(s) set;
a machine readable storage device; and
computer code stored on the machine readable storage device, with the computer code including instructions for causing the processor(s) set to perform operations including the following:
receiving an original image data set corresponding to an original image for electronic transmission that will include application of a transformation to compress data as the data is being transmitted,
determining, with a computer vision machine learning (ML) model, an original set of ML output values that represent a human understanding of information present in the original image,
applying the transformation to the original image data set to obtain a test transmission image data set corresponding to a transmission image,
determining, with the computer vision machine learning (ML) model, a transmission test set of ML output values that represent a human understanding of information present in the transmission image, and
comparing the transmission test set of ML output values with the original set of ML output values for differences to determine that an unacceptable degree of information loss is occurring in the electronic transmission of images used in connection with the computer vision ML model.

14. The CS of claim 13, wherein the computer code further includes instructions for causing the processor(s) set to perform the following operations:
responsive to determining that an unacceptable degree of information loss is occurring in the electronic transmission of images used in connection with the computer vision ML model, transmitting the original image to a receiver electronic device through the electronic transmission, where the transformation to compress data is disabled for transmission of the original image.

15. The CS of claim 13, wherein the computer code further includes instructions for causing the processor(s) set to perform the following operations:
responsive to determining that an unacceptable degree of information loss is occurring in the electronic transmission of images used in connection with the computer vision ML model, determining a set of modifications to the original image based, at least in part, on differences between the original set of ML output values and the test set of ML output values.

16. The CS of claim 15, wherein the computer code further includes instructions for causing the processor(s) set to perform the following operations:
generating a modified image for electronic transmission corresponding to the original image transformed with the set of modifications; and
transmitting the modified image to a receiver electronic device through electronic transmission.

17. The CS of claim 15, wherein the set of modifications are selected from the group consisting of: scaling up segments of text to a larger font size when data values corresponding to those segments of text in the original image and the test image are determined differently by the computer vision ML model, magnifying portions of the original image when data values corresponding to those portions of the original image and test image are determined differently by the computer vision ML model, and annotating text upon elements in the image when data values corresponding to those elements of the original image and the test image are determined differently by the computer vision ML model.

18. The CS of claim 13, wherein:
the original image corresponds to medical information; and
the electronic transmission corresponds to a fax transmission.
